(19) United States
(12) Patent Application Publication
(10) Pub. No.: US 2013/0281386 A1
(43) Pub. Date: Oct. 24, 2013

(54) Glufosfamide Combination Therapies for Cancer

(71) Applicants: Forrest H. Anthony, Villanova, PA (US); Edwin J. Thomas, Moorestown, NJ (US); Matthew Parris, Plainsboro, NJ (US)

(72) Inventors: Forrest H. Anthony, Villanova, PA (US); Edwin J. Thomas, Moorestown, NJ (US); Matthew Parris, Plainsboro, NJ (US)

(73) Assignee: Eleison Pharmaceuticals LLC, Princeton, NJ (US)

(21) Appl. No.: 13/599,684

(22) Filed: Aug. 30, 2012

Related U.S. Application Data
(60) Provisional application No. 61/687,114, filed on Apr. 19, 2012.

(51) Int. Cl. A61K 31/7028; A61K 31/7042; A61P 35/00; A61K 31/7034

(52) U.S. Cl. 514/25

(57) ABSTRACT

The invention provides compositions and methods for treating cancer with glufosfamide in combination with an inhibitor of sodium-glucose transporter type 2 (SGLT2) to block the uptake of glucose in the proximal tubules of the kidneys to decrease renal toxicity. The invention relates to the fields of biomedicine, pharmacology, and molecular biology.

### CROSS-REFERENCE TO RELATED APPLICATIONS

This application claims priority to U.S. Provisional Patent Application No. 61/687,114, filed Apr. 19, 2012, which is incorporated herein by reference.

### FIELD OF INVENTION

The present invention relates generally to methods for increasing the Therapeutic Index of glucose conjugated drugs such as glufosfamide by reducing its renal toxicity using co-administration of an inhibitor of glucose reabsorption in the kidney tubules. Blocking glucose reabsorption with a SGLT2 inhibitor reduces the renal toxicity of glufosfamide, thus making it more effective in the treatment of cancer, because either higher doses can be given, or less toxicity is associated with an effective dose. In particular, this invention relates to administration of glufosfamide in combination with SGLT2 inhibiting drugs. The invention relates to the fields of biomedicine, pharmacology, and molecular biology.

### BACKGROUND

Glufosfamide, also known as β-D-glucosyl-ifosfamide mustard or glc-IPM, is a prodrug of an alkylating agent, isophosphoramide mustard. Glufosfamide has been used in the clinic as an investigational agent in the treatment of cancer. See U.S. Pat. Nos. 5,622,936 and 6,489,302 and PCT Publication Nos. WO 05/076888, WO 06/071955, WO 06/122227, and WO 07/035,961, each of which is incorporated herein by reference. In contrast to the alkylating agent prodrug ifosfamide, metabolism of glufosfamide does not systemically release the toxic metabolite acrolein, and also produces less of the toxic metabolite chloroacetaldehyde. Glufosfamide was recently tested in a Phase III clinical trial for the treatment of pancreatic cancer; it was administered intravenously over six hours, every three weeks. Although the data indicated that the drug was having an anti-cancer effect, the trial did not meet its primary endpoint with statistical significance p<0.05. One hypothesis is that the main side effect of the drug is kidney damage, also known as proximal tubular acidosis, or “PTA”. PTA limits the dosage amount and frequency of the glufosfamide anticancer treatment. A treatment that would protect the kidney from glufosfamide would make glufosfamide a more effective anticancer agent by making higher drug exposure possible without dangerous kidney damage. There is a clinical need for new therapies that limit the renal side effects of glufosfamide so that it might be used safely at higher doses to more effectively treat cancer.

As is known in the art, specific drugs may be co-administered with chemotherapeutics to reduce chemotherapy-related toxicity, thereby allowing the chemotherapy to be safely given at higher and/or more effective doses. An example of this is mercaptoethane sulfonate-sodium (“MESNA”), a drug given with ifosfamide chemotherapy, to prevent bleeding from the cells lining the urinary bladder, which otherwise occurs frequently with ifosfamide. Administration of MESNA avoids this complication and allows higher and/or longer dosing with ifosfamide, and a greater antitumor efficacy.

Sodium-glucose transporter-2 mediated reabsorption of glucose-conjugated chemotherapeutic drugs occurs in the proximal tubules of the kidney. This causes a toxic effect, or nephrotoxicity, which can severely damage the kidneys, limiting the amount of drug that can be tolerated. Generally, chemotherapeutic drugs are administered at the highest doses tolerated, because the efficacy against cancer increases with dose and duration of dose.

Sodium-glucose transporter-2 (“SGLT2”) is a protein found exclusively localized in the kidney. SGLT2 is responsible for approximately 90% of the glucose reabsorption in the kidney. Glucose reabsorption is a usual function in normal physiology, so that glucose is not lost in the urine. Recently, a number of specific inhibitors of SGLT2 have been developed for potential use to reduce plasma glucose levels in diabetes mellitus type 2 (“DM2”). As reabsorption of glucose is blocked in the kidney tubules by the SGLT2, extra glucose is excreted. This lowers blood glucose levels and is a therapeutic treatment for DM2. It appears that these specific inhibitors are safe and effective in blocking glucose reabsorption in the kidneys.

The present invention combines the glucose reabsorption blocking mechanism of SGLT-2 inhibitors with the anticancer treatment effect of glufosfamide. Accordingly, the present invention satisfies an unmet need of treating cancer with glufosfamide, without damaging the kidneys, by providing novel combination therapies of glufosfamide and an SGLT2 inhibitor, as summarized below and described in detail herein.

### SUMMARY

In one embodiment, a method of treating a patient diagnosed with cancer is provided, where the method includes 1) administering an agent that inhibits the reabsorption of glucose by the kidneys to the patient, and 2) administering a therapeutically effective amount of a glucose-conjugated chemotherapeutic agent to the patient.

In one embodiment, a combination therapy is provided that includes (i) an inhibitor of the kidney glucose transporter type 2 (SGLT2) such as dapagliflozin; and (ii) an effective amount of a glucose-conjugated chemotherapeutic drug. In one embodiment, the glucose-conjugated chemotherapeutic drug is Glufosfamide.

### DETAILED DESCRIPTION

It was recognized that a sodium-glucose transporter type 2 inhibitor can be used to protect against renal damage associated with the chemotherapeutic drug glufosfamide, improving its therapeutic index and utility in cancer therapy. This therapy combines the glucose reabsorption blocking mechanism of SGLT2 inhibitors with the anticancer treatment effect of glufosfamide. This treatment protects the kidneys against toxic effects of glufosfamide, which are mediated by SGLT2 transporters in the proximal tubules of the kidneys.

Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

“Administering” or “administration” of a drug to a patient (and grammatical equivalents of this phrase) refers to direct administration, which may be administration to a patient by a medical professional or may be self-administration, and/or indirect administration, such as the act of prescribing a drug. For example and without limitation, a physician who instructs a patient to self-administer a drug and/or provides a patient with a prescription for a drug is, for purposes of the present invention, “administering” the drug to the patient.

“Agent that inhibits the uptake of glucose” means any agent that is capable of blocking the reabsorption of glucose by the kidneys.

“Brush border cells” refers to the epithelial cells that line the proximal tubule in the kidney. These cells have microvilli on their luminal surface. SGLT2 transport proteins are largely responsible for the physiological reabsorption of glucose, but also for the transport of glufosfamide into these cells, which causes cell damage and loss of kidney function.

“Dapagliflozin” refers to one of several drugs in the class that inhibits subtype 2 of the sodium-glucose transport system [SGLT2], which inhibition causes blood glucose, or glucose-conjugated drugs, to be eliminated in the urine instead of being actively reabsorbed by the kidneys.

“Diabetes mellitus” refers to a group of metabolic diseases characterized by high serum glucose levels, which can result, for example, from defects in insulin secretion, action, or both.

“Diabetic patient” refers to a patient suffering from diabetes mellitus.

“Effective amount” or “therapeutic amount” or “therapeutically effective amount” refers to an amount effective, at dosages and for periods of time necessary, to achieve a desired therapeutic result. A therapeutically effective amount for glufosfamide may vary according to factors such as the disease state, age, sex, and weight of the individual patient, and the ability of the glufosfamide to elicit a desired response in the individual. A therapeutically effective amount is also one in which any toxic or detrimental effects of the treatment are outweighed by the therapeutically beneficial effects. The therapeutically effective amount for tumor therapy may also be measured by its ability to stabilize the progression of disease, or to reduce the size of tumors, or to cause their complete disappearance. The ability of the treatment to inhibit cancer may be evaluated in an animal model system predictive of efficacy in human tumors. Alternatively, this property of a composition may be evaluated by examining the ability of the compound to inhibit cell growth or induce apoptosis by in vitro assays known to one of ordinary skill in the art. The effective amount (dose) may decrease tumor size, or otherwise ameliorate symptoms in a patient. The skilled practitioner would be able to determine such amounts based on patient's size, severity of the patient's symptoms, and the particular route of administration.

“Glufosfamide” or “Glufos™” refers to the antitumor drug β-D-glucosyl-ifosfamide mustard (glc-IPM), which is an alkylating agent used for the treatment of cancer (see U.S. Pat. No. 5,622,936, the entire contents of which are incorporated herein by reference).

“Nephrotoxicity” or “renal toxicity” refers to a poisonous effect that a substance has on the kidneys. “Nephrotoxic” substances may cause renal (kidney) failure, which can be temporary or permanent.

“Patient” or “Subject” refers to a mammal in need of treatment for cancer or, in some embodiments, for a hyperproliferative disease other than cancer. Generally, the patient or subject is a human. In other embodiments of the invention, however, the patient or subject is a non-human mammal, such as a non-human primate, a dog, cat, cow, horse, rabbit, pig, or the like. In other embodiments of the invention, the patient or subject is an animal such as a mouse or rat, such as an animal commonly used in screening, characterizing, and evaluating drugs and therapies.

“Proximal Tubular Acidosis” or “PTA” refers to an abnormal condition characterized by excessive acid accumulation and bicarbonate excretion. PTA is caused by the defective reabsorption of bicarbonate in the proximal tubules of the kidney and the resulting flow of excessive bicarbonate into the distal tubules, which normally secrete hydrogen ions. PTA is often the result of damage to the brush border cells that line the proximal tubules. An example of this type of damage is that seen by the administration of glufosfamide.

“Sodium-glucose transporter type 2” or “SGLT2” refers to a transporter protein that is responsible for approximately 90% of the active transport of glucose in the proximal tubule of the kidney.

“Sodium-glucose transporter type 2 inhibitor” or “SGLT2 inhibitor” refers to an agent that blocks reabsorption of glucose by the kidneys.

“Therapeutic Index” refers to the ratio of the toxic dose to the effective dose for any drug. Accordingly, increasing the therapeutic index of a drug is useful in making the drug either safer or more effective.

“Treatment” or “therapy” refers to a method for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delaying or slowing of disease progression, amelioration or palliation of the disease state, and remission (whether partial or total).

“Treatment” can also mean prolonging survival as compared to expected survival in the absence of receiving treatment.

The present invention relates to co-administration of an SGLT2 inhibitor with a chemotherapeutic drug in order to reduce the kidney damage caused by the chemotherapeutic drug. Reducing the kidney damage allows for greater safety and/or allows a higher dose to be used, which may be more effective than a lower or more infrequent dose. Glufosfamide, whose molecular structure includes a glucose molecule, is transported into the brush border cells by SGLT2 transporters, and then is activated and damages the brush border cells which cause PTA in the patient, which often limits the dose or frequency of dosing for a specific patient. This nephrotoxicity is blocked by the SGLT2 inhibitors, which allows the glufosfamide to be given at higher doses, or more often, and thus more effective in treating cancer in the patient.

In some embodiments, the chemotherapeutic drug is glufosfamide. Glufosfamide is an experimental cytotoxic chemotherapeutic drug that appears to be active in a number of solid tumor indications, such as pancreatic cancer, soft tissue sarcomas, and colorectal cancer. However, treatment with glufosfamide causes side effects. One example is toxicity that causes damage to the proximal tubule of the kidney, caused by active reabsorption of the glucose containing drug into the cells lining the proximal tubule, and causing damage to these cells. If the toxicity is severe enough, treatment with glufosfamide must be reduced or terminated in its entirety.

Similar to glucose, reabsorption of glufosfamide in the proximal tubules is specifically mediated by SGLT-2, which is expressed in the kidney tubule cells. An SGLT-2 inhibitor blocks the uptake of glufosfamide in the kidney, thus protecting the kidney against tubular cell damages. The combination of the SGLT-2 inhibitor and glufosfamide allows a higher dose intensity of glufosfamide, without toxicity concerns. Therefore, the efficacy and Therapeutic Index of glufosfamide against cancer is increased. Glufosfamide requires other glucose transporters expressed by tumor cells to achieve tumor uptake and tumor cell death. However, these other tumor cell glucose transporters are not affected by SGLT2 specific inhibitors because SGLT2 occurs only in the kidneys.

In one embodiment of the invention, glufosfamide and an agent which inhibits the uptake of glucose by the kidneys are administered in combination to a patient in need of treatment for cancer. In some embodiments, glufosfamide and the agent which inhibits the uptake of glucose by the kidneys are administered in combination to a patient in need of first-line treatment for cancer. In some embodiments, glufosfamide and an SGLT2 inhibitor would be administered in combination with other chemotherapeutics agent(s).

In one embodiment of the invention, glufosfamide and a sodium-glucose transporter-2 (SGLT2) inhibitor are administered in combination to a patient in need of treatment for cancer. In one embodiment, the inhibitor of SGLT2 is dapagliflozin.

In one embodiment of the invention, glufosfamide and an inhibitor of SGLT2 are administered in combination to a patient in need of first-line treatment for cancer, in combination with one or more other cancer therapeutic agents. In one embodiment, the inhibitor is dapagliflozin.

In another embodiment of the invention, glufosfamide and an agent which inhibits the uptake of glucose by the kidneys, one example of such agent is an SGLT2 inhibitor, and one example of such an inhibitor is dapagliflozin, are administered in combination to a patient in need of second-line treatment for cancer, or post-second-line treatment for cancer.

In an aspect of the invention, glufosfamide and an agent which inhibits the uptake of glucose by the kidneys, one example of such agent is an SGLT2 inhibitor, and one example of such an inhibitor is dapagliflozin, are administered in combination to a subject in need of second-line treatment for cancer, or post second-line treatment of cancer, in combination with one or more other cancer therapeutic agents.

In an aspect of the invention, glufosfamide and an agent which inhibits the uptake of glucose by the kidneys, one example of such agent is an inhibitor of SGLT2, and one example of such an inhibitor is dapagliflozin, are administered in combination to a subject in need of treatment for gemcitabine-refractory cancer.

In an aspect, the invention provides a treatment method in which glufosfamide, in combination with an agent which inhibits the uptake of glucose by the kidneys, one example of such agent is an inhibitor of SGLT2, and one example of such an inhibitor is dapagliflozin, which is administered according to a schedule or administration regimen discovered to be particularly effective for treatment of cancer.

In treatment regimens in which glufosfamide and an agent which inhibits the uptake of glucose by the kidneys, one example of such agent is an inhibitor of SGLT2, and one example of such an inhibitor is dapagliflozin, are administered in combination, they can be administered in any order. The glucose reabsorption block must be in effect during the administration of glufosfamide. The glufosfamide should not be administered without a full inhibition of glucose reabsorption, and conversely there is no need for SGLT2 inhibitor unless glufosfamide is also present, or to be administered momentarily.

When two or more drugs are administered in combination, a variety of schedules can be used. In certain embodiments, glufosfamide is administered on the same day concurrent with, commenced before, or commenced after administration of agent which inhibits the uptake of glucose by the kidneys, one example of such agent is an inhibitor of SGLT2, and one example of such an inhibitor is dapagliflozin. It will be understood that other schedules can be used as determined by a physician. As is understood in the art, treatment with cancer therapeutic drugs can be suspended temporarily if toxicity is observed, or for the convenience of the patient, without departing from the scope of the invention, and then resumed.

### Therapeutic Methods of the Invention

In one aspect, the present invention provides a method of treating a patient diagnosed with cancer, by

1) verify that the patient is not hyperglycemic and has not taken insulin;
2) administering an agent that inhibits the reabsorption of glucose by the kidneys to said patient, and
3) administering a therapeutically effective amount of glufosfamide to the patient.

If the patient is under prior or current treatment with insulin, step 2 is delayed until it is determined that administration of glufosfamide is acceptable. For example, if the patient was given a dose of insulin on Day 1, they may not start step 2 until Day 2. Once it is determined that the patient has not taken insulin within at least 24 hours of administration of glufosfamide, the patient is administered an agent that inhibits the uptake of glucose by the kidneys. The patient is monitored to determine levels of serum glucose. Once it is determined that glucose levels are acceptable, the patient is administered a therapeutically effective amount of glufosfamide.

The agent that inhibits the uptake of glucose by the kidneys can be administered prior to and/or contemporaneously with the administration of glufosfamide (“glufosfamide Day 1”). As used in this context, “contemporaneously” can mean the two drugs are administered on the same day, or on consecutive days, or within a week of one another. It will be understood that use of the word “or” in this context does not exclude combinations, such as administration the day before and the same day as glufosfamide administration.

In one approach the agent that inhibits the uptake of glucose by the kidneys is administered as part of a treatment regimen contemporaneously with each of multiple administrations of glufosfamide (e.g., at least 2, 3, 4, 5, 6, 7, 8, 9, 10, 11, 12, 13 or 14 rounds of administration). In one approach the agent that inhibits the uptake of glucose by the kidneys is administered as part of a treatment regimen contemporaneously with each administration of glufosfamide.

In one approach the agent that inhibits the uptake of glucose by the kidneys is administered as part of a treatment regimen contemporaneously with each administration of glufosfamide. For example, the agent that inhibits the uptake of glucose from the kidneys may be given daily throughout the course of glufosfamide treatment, or daily at least through several cycles.

In some embodiments, the SGLT2 inhibitor is administered contemporaneously with the glufosfamide. In some embodiments, the SGLT2 is dapaglifozin, which is administered with the glufosfamide.

Again, it will be understood that description of certain administration schedules is not intended to be limiting, and that, for example, combinations of administration schedules described herein.

In some embodiments, when the agent that inhibits the uptake of glucose by the kidneys is administered after the initiation of glufosfamide therapy it can be administered daily, on the same days glufosfamide is administered (e.g., once every 21 days if a three-week treatment cycle is used), one day before and/or one day after glufosfamide treatment, or according to another schedule. The agent that inhibits the uptake of glucose by the kidneys therapy can continue for the duration of glufosfamide treatment (e.g., up to 42 weeks, using the treatment cycle described above) or for a shorter period. In some embodiments, the agent is an SGLT2 inhibitor that blocks uptake of glucose during glufosfamide administration and when glufosfamide can be detected in the bloodstream. In some embodiments, the administration of the agent may be stopped once glufosfamide is no longer detectable in the bloodstream, e.g. approximately six (6) hours after administration of the glufosfamide has stopped.

In one embodiment of the methods of the invention, the cancer patient is administered an agent that inhibits the uptake of glucose by the kidneys at least during the period of time in which the patient is being administered glufosfamide. Often, in accordance with the methods of the invention, agent that inhibits the uptake of glucose by the kidneys is administered throughout the period of glufosfamide administration and, usually, prior to initiation of glufosfamide treatment.

In one embodiment, the agent that inhibits the uptake of glucose by the kidneys is an inhibitor of sodium-glucose transporter-type 2. In one embodiment, the agent that inhibits the uptake of glucose by the kidneys is dapagliflozin. In other embodiments, the agent that inhibits the uptake of glucose by the kidneys is selected from the group consisting of SGLT2 inhibiting drugs. Examples of SGLT2 inhibiting drugs may include those found in Table 1 below:

TABLE 1
Examples of SGLT2 Inhibitors
DRUG NAME

T-1095
AVE2268
Remogliflozin etabonate
Sergliflozin
Dapagliflozin
JNJ-28431754/TA-7284 [Canagliflozin]
BI 10773 [Empagliflozin]
From: The Journal of Clinical Endocrinology & Metabolism Jan. 1, 2010 vol. 95 no. 1 34-42

In one aspect, the present invention provides a method of treating a patient diagnosed with cancer, by 1) determining whether the patient is receiving insulin or is hyperglycemic, and, 2) if it is determined the patient is not receiving insulin and is not hyperglycemic, then administering an inhibitor of sodium-glucose transporter-2, and 3) administering a therapeutically effective amount of glufosfamide to the patient.

In one aspect, the present invention provides a method of treating a patient diagnosed with cancer, by 1) determining whether the patient is receiving insulin or is hyperglycemic, and, 2) if it is determined the patient is receiving insulin, discontinuing the insulin therapy, initiating therapy with an inhibitor of sodium-glucose transporter-2, and when fasting glucose levels are in a normal range 3) administering a therapeutically effective amount of glufosfamide to the patient. In an embodiment the inhibitor of sodium-glucose transporter-2 is administered prior to the initiation of glufosfamide therapy. In one embodiment, the inhibitor of sodium-glucose transporter-2 is administered contemporaneously with administration of glufosfamide therapy.

In one aspect, the present invention provides a method of treating a patient diagnosed with cancer, by 1) determining whether the patient is receiving insulin or is hyperglycemic, and, 2) if it is determined the patient is receiving insulin, discontinuing insulin therapy and initiating therapy with dapagliflozin, and 3) administering a therapeutically effective amount of glufosfamide to the patient. In one embodiment, the dapagliflozin is administered prior to the initiation of glufosfamide therapy. In one embodiment, the dapagliflozin is administered contemporaneously with administration of glufosfamide therapy.

In one embodiment, if it is determined that a patient diagnosed with cancer is receiving insulin, then the insulin therapy is discontinued, prior to administration of glufosfamide. In one embodiment the dapagliflozin is administered to the patient in combination with glufosfamide. In one embodiment, the dapagliflozin is administered prior to the administration of glufosfamide.

### A. Administration Cycles

Cancer chemotherapy treatment typically involves multiple “rounds” or “cycles” of drug administration, where each cycle comprises administration of the drug one or more times according to a specified schedule (e.g., daily; once per week for two or more weeks; multiple times a week either on consecutive days or non-consecutive days; once every cycle, which may be a day, week, or month, for example; multiple times every cycle [for example and without limitation every three weeks for three consecutive days], wherein each cycle ranges from 1 day to 1 week up to several weeks, such as 2, 3, 4, 5, 6, 7, or 8 weeks). For example and without limitation, chemotherapeutic drugs can be administered for from 1 to 8 cycles, or for more cycles (i.e., a longer time period). As is understood in the art, treatment with anticancer therapeutic drugs can be suspended temporarily if toxicity is observed, or for the convenience of the patient, without departing from the scope of the invention, and then resumed.

In one embodiment of the invention, glufosfamide is administered for 1, 2, 3, 4, 5, 6, 7, 8, or more than 8 dosage cycles, and each cycle involves the administration by infusion of glufosfamide in the range of:

a) about 1.0 to about 8.0 g/m²; about 1.0 to about 6.0 g/m²; about 1.5 to about 4.5 g/m²; about 4.5 to about 8.0 g/m²; about 4.5 to about 6.0 g/m²; or about 4.5 to about 5.0 g/m² or over an infusion period of 1-6 hours once every week; b) about 5.0 to about 12.0 g/m²; about 6.0 to about 10.0 g/m²; about 6.5 to about 9.5 g/m²; or about 7 to about 9.0 g/m²; over an infusion period of 1-6 hours once every three weeks; c) about 1.0 to about 3.0 g/m², about 1.5 to about 3.0 g/m² or about 1.5 to about 2.0 g/m² over an infusion period of 1-6 hours for three consecutive days (days 1, 2 and 3) every three weeks; d) about 1.0 to about 3.0 g/m², about 1.5 to about 3.0 g/m² or about 1.5 to about 2.0 g/m² over an infusion period of 1-6 hours for three consecutive days (days 1, 2 and 3) every three weeks; e) about 1.0 to about 2.0 g/m² or about 1.5 to about 2.0 g/m² over an infusion period of 1-6 hours once per week; or
f) about 1.0 to about 12.0 g/m²; about 5.0 to about 9.0 g/m²; or about 6 to about 8 g/m² over an infusion period of 1-6 hours once every four weeks.

In one embodiment, glufosfamide is administered for 1, 2, 3, 4 or more than 4 dosage cycles, wherein each cycle is a seven-week cycle. In one embodiment, glufosfamide is administered for 1, 2, 3, 4, 5, 6, or more than 6 dosage cycles, wherein each cycle is a three-week cycle. In one embodiment, glufosfamide is administered for 1, 2, 3, 4, 5, 6, or more than 6 dosage cycles, wherein each cycle is a four-week cycle. In one embodiment, glufosfamide is administered weekly in the range of 1.0 to about 3.0 g/m2, for example and without limitation on Days 1 and 8 of a 21 day cycle; on Days 1, 8, and 15 of a 28 day cycle; or Days 1, 8, and 15 of a 21 day cycle. As used in this context, an “infusion period of 1-6 hours” includes without limitation, an infusion period of about 1, about 2, about 3, about 4, about 5, and about 6 hours.

### B. Treatment Combinations

During chemotherapy treatment of cancer, two, three, or four anti-cancer drugs can be administered to a patient “in combination” by administering them as part of the same course of therapy. A course of therapy refers to the administration of combinations of drugs believed by the medical professional to work together additively, complementarily, synergistically, or otherwise to produce a more favorable outcome than that anticipated.

For illustration and not limitation, the administration of glufosfamide and various other anti-cancer drugs for treatment of cancer is found in U.S. Patent Application Nos. 61/027,768, filed 11 Feb. 2008; 60/991,660, filed 30 Nov. 2007; 60/952,686, filed 30 Jul. 2007; 60/915,882, filed 3 May 2007; and 60/910,403, filed 5 Apr. 2007, and PCT Pub. Nos. WO 05/076888, WO 06/071955, WO 06/122227, and WO 07/035,961, each of which is incorporated herein by reference. The administration and dosing schedules described in these publications and applications are suitable for use in the methods of the present invention.

C. Cancers Treatable in Accordance with the Methods of the Invention

In one embodiment, the present invention provides methods for treating pancreatic cancer. In another embodiment, the cancer treated is selected from a primary pancreatic cancer, metastatic pancreatic cancer, and gemcitabine resistant pancreatic cancer (primary and metastatic). Chemotherapy-resistant pancreatic cancers (see, e.g., Araneo et al., 2003, Cancer Invest. 21:489-96; Kozuch et al., 2001, The Oncologist 6:488-95; Noble and Goa, 1997, Drugs 54: 44772N; Stephens et al., 1998, Oncol. Nurs. Forum 25:87-93; Burris and Storniolo, 1997, Eur. J. Cancer 33: Suppl 1:S1822; Rothenberg et al., 1996, Ann. Oncol. 7:347-53, each of which is incorporated herein by reference) can be treated using the methods disclosed herein. In one embodiment of the invention, serum carbohydrate 19-9 is used as a marker for evaluating the response to such glufosfamide therapy in pancreatic cancer (Ziske et al., 2003, Br. J. Cancer, 89:1413-17, incorporated herein by reference).

In various embodiments, the methods of the present invention can be used for the treatment of any cancer, including but not limited to pancreatic cancer, colorectal cancer, soft tissue sarcomas, ovarian cancer, lung cancer, breast cancer, glioblastoma, skin cancer, bone cancer, liver cancer, prostate cancer, sarcoma, non-Hodgkin's lymphoma, kidney cancer, gall bladder cancer, stomach cancer, brain cancer.

In general, the methods of the present invention can be used for treatment of any cancer. In various embodiments, the cancer treated is selected from the group consisting of cancer of the adrenal gland, bone, brain, breast, bronchi, colon and/or rectum, gallbladder, head and neck, kidneys, larynx, liver, lung, neural tissue, pancreas, prostate, parathyroid, skin, stomach, and thyroid. In other embodiments, the cancer treated is selected from the group consisting of acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteosarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, small cell lung cancer, topical skin lesion, reticulum cell sarcoma, and Wilm's tumor.

All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

### EXAMPLES

### Example 1

A patient having pancreatic cancer is given an oral dose of dapagliflozin (an SGLT2 inhibitor) to cause complete inhibition of SGLT2 function in the kidneys, at least two hours before starting an intravenous dose of glufosfamide on Day 1. The usual dose for glufosfamide alone would be 4500 mg/m² of glufosfamide and is administered over a six hour period, every 21 days. However, with the protective effects of a SGLT2 blocking drug, the tolerated dose is much higher, i.e. 6000 mg/m² or up to 12,000 mg/m² q 3 wks (or more often). Renal function in all patients is carefully monitored. These higher or more frequent doses may be tolerated without renal side effects, as long as the SGLT2 drug is in effect. Therefore the drug is more effective against pancreatic or other types of cancer. At higher doses, and without kidney toxicity, glufosfamide is more likely to cause cancer regression or stabilization, which is a clinical benefit.

1. A combination therapy comprising:
(i) an inhibitor of a kidney glucose transporter protein
(ii) an effective amount of a glucose-conjugated chemotherapeutic drug such as glufosfamide.

2. The therapy of claim 1, wherein said kidney glucose transporter inhibitor is an SGLT2 inhibiting drug.

3. The therapy of claim 2, wherein said SGLT2 inhibitor is selected from the group consisting of dapagliflozin, canagliflozin, ipragliflozin, tofogliflozin, BI-10773, sergliflozin etabonate, remogliflozin etabonate, and combinations thereof.

4. The therapy of claim 2, wherein said sodium glucose transporter type 2 inhibitor is dapaglifozin.

5. The therapy of claim 1, wherein said chemotherapeutic drug is selected from the group consisting of: any cytotoxic, cytostatic agent, or molecularly targeted anticancer agent conjugated with glucose.

6. The therapy of claim 1, wherein said chemotherapeutic drug is glufosfamide.

7. A method of treating cancer in a patient comprising administering a therapeutically effective amount of glufosfamide in combination with sodium-glucose transporter type 2 inhibitor to a patient in need of a cancer treatment.

8. The method of claim 7, wherein said cancer is pancreatic adenocarcinoma.

9. The method of claim 7, wherein said sodium-glucose transporter type 2 inhibitor is selected from the group consisting of dapagliflozin, canagliflozin, ipragliflozin, tofogliflozin, BI-10773, sergliflozin etabonate, remogliflozin etabonate, and combinations thereof.

10. The method of claim 9, wherein said sodium-glucose transporter type 2 inhibitor is dapagliflozin.

12. A method of treating a patient diagnosed with cancer, said method comprising the steps of:
(i) administering an agent that inhibits the absorption of glucose by the kidneys to said patient; and
(ii) administering a therapeutically effective amount of glucose-conjugated chemotherapeutic agent to said patient.
